(19) United States
(12) Patent Application Publication
(10) Pub. No.: US 2026/0116746 A1
(43) Pub. Date: Apr. 30, 2026

(54) Nuclear Driven Hydrothermal Decomposition of an Inert Sodium Salt for the Production Of Hydrogen

(71) Applicant: NuScale Power, LLC, Corvallis, OR (US)

(72) Inventors: Francis Y. TSANG, Bellingham, WA (US); Luis DEPAVIA, Corvallis, OR (US); José N. REYES, JR., Corvallis, OR (US)

(73) Assignee: NuScale Power, LLC, Corvallis, OR (US)

(21) Appl. No.: 19/041,553

(22) Filed: Jan. 30, 2025

Related U.S. Application Data
(60) Provisional application No. 63/626,789, filed on Jan. 30, 2024.

(51) Int. Cl. C01B 3/02; B01D 53/047; B01J 8/00; B01J 8/08; C01B 3/56; C01B 32/50; C01D 1/04; C01D 7/00; C25B 1/16; C25B 15/08; G21D 1/02; H01M 8/0612

(52) U.S. Cl. CPC C01B 3/02; B01D 53/047; B01J 8/0045; B01J 8/085; B01J 8/087; C01B 3/56; C01B 32/50; C01D 1/04; C01D 7/00; C25B 1/16; C25B 15/081; G21D 1/02; H01M 8/0612; B01D 2256/16; B01D 2257/504; B01J 2208/00168; B01J 2208/00769; C01B 2203/042; C01B 2203/0475; C01B 2203/066; C01B 2203/0894; C01B 2203/1211

(57) ABSTRACT

Methods and systems for hydrogen production from inert sodium salts are described herein. In an example method, steam is generated by a nuclear reactor power plant system. The steam is applied to sodium formate to facilitate one or more thermal and/or hydrothermal decomposition processes, thereby generating hydrogen. In the example method, sodium formate is generated by combining sodium hydroxide generated by an electrolysis process with sodium carbonate and/or sodium bicarbonate generated by a carbon capture process. Embodiments can be used to supply hydrogen storage facilities and/or for energy production.

### CROSS-REFERENCE TO RELATED APPLICATIONS

This application claims the benefit of U.S. Provisional Patent Application No. 63/626,789, filed Jan. 30, 2024, and titled “Nuclear Driven Hypothermal Decomposition of Inert Sodium Salts for the Production of Hydrogen,” which is incorporated herein by reference in its entirety.

### BACKGROUND

Hydrogen is considered a very important energy carrier that can be used to store and transform into different energy products. In 2022, roughly 92 million metric tons of Hydrogen (H₂) were produced globally. More than 95% of produced Hydrogen is generated through fossil fuels by: (1) steam-methane reforming of natural gas; (2) oxidation of hydrocarbons; (3) coal gasification; or (4) by biomass gasification. These processes generate very large Carbon Dioxide (CO₂) footprints that have been identified as a major source of greenhouse gases and contribute to climate change and global warming. Furthermore, transportation and delivery of Hydrogen from the point of production to the point of use is conventionally accomplished using pipelines and/or using large insulted cryogenic tankers, further increasing the greenhouse gas footprint of the Hydrogen production process. Hydrogen is conventionally stored either as a compressed gas or as a liquid for delivery by cryogenic tankers. Recently, production of Hydrogen via electrolysis of water has been introduced as an alternative Hydrogen production process with a lower greenhouse gas footprint.

An energy imbalance market (“EIM”) is a means of supplying energy when and where it is needed to balance fluctuations in energy demand (e.g., peak times vs. off-peak times) and subsequent fluctuations in energy production demand (e.g., energy production demand during peak times vs. energy production demand during off-peak times). For example, highly populated areas with hot climates may experience a large increase in energy demand in the evenings caused by a large number of consumers getting home from work and turning on air conditioning units. Similarly, the same highly populated areas with hot climates may see large decreases in energy demand in the mornings caused by the large number of consumers turning off air conditioners and going to work. The times of high energy demand, or “peak times,” and times of low energy demand, or “off-peak times,” may be anticipated and planned for. As a result of the fluctuations between peak times and off-peak times, energy providers (e.g., nuclear, solar, natural gas, fossil fuel, etc.) may experience similar fluctuations in the demand for energy production (e.g., higher energy production demand during peak times than during off-peak times). Peaker plants are often employed to support the additional energy demand during peak times. Peaker plants are typically fueled by natural gas, diesel, or other fossil fuels, and emit high amounts of greenhouse gases.

To increase productivity and efficiency, and to reduce greenhouse gas emissions, for energy producers experiencing fluctuating energy production demand, new processes, systems, and methods are needed. Energy from power generation plants, such as, for example, nuclear reactors and/or renewable sources, can be diverted to produce Hydrogen as an energy carrier for short-term storage during off-peak hours to support the EIM.

The terminology used in the Detailed Description presented below is intended to be interpreted in its broadest reasonable manner, even though it is being used in conjunction with a detailed description of certain specific embodiments of the disclosure. Certain terms may even be emphasized below; however, any terminology intended to be interpreted in any restricted manner will be overtly and specifically defined as such in this Detailed Description section. The headings provided herein are for convenience only and should not be construed as limiting the subject matter disclosed.

### BRIEF DESCRIPTION OF THE DRAWINGS

FIG. 1 schematically illustrates a representation of an integrated energy system that includes a small modular nuclear reactor (SMR) system integrated with a Hydrogen production system and a hydrogen fuel cell.

FIG. 2 illustrates a steady-state Hydrogen production process utilizing Sodium Formate and an electrochemical device.

FIG. 3 illustrates a steady-state Hydrogen production process utilizing Sodium Formate and a hydrogen fuel cell.

FIG. 4 illustrates in-situ and on-demand Hydrogen production system to support emergency and limited energy imbalance market (EIM) using Sodium Formate.

FIG. 5 schematically illustrates a representative schematic diagram of a Hydrogen extraction reactor during steady state operations for the hydrothermal decomposition of Sodium Formate.

FIG. 6 schematically illustrates a representative schematic diagram of a Hydrogen extraction reactor during steady state operations for a combination of thermal decomposition and hydrothermal decomposition of Sodium Formate.

FIG. 7 illustrates a Hydrogen production process using multiple Sodium Formate production systems simultaneously.

FIG. 8 illustrates a flow diagram of an example process associated with utilizing a Hydrogen extraction reactor for production of Hydrogen from Sodium Formate.

FIG. 9 is a partially schematic, partially cross-sectional view of a nuclear reactor system configured in accordance with embodiments of the present technology.

FIG. 10 is a partially schematic, partially cross-sectional view of a nuclear reactor system configured in accordance with additional embodiments of the present technology.

FIG. 11 is a schematic view of a nuclear power plant system including multiple nuclear reactors in accordance with embodiments of the present technology.

### DETAILED DESCRIPTION

The present disclosure is directed to Hydrogen (H₂) generation from inert sodium salts, such as from Sodium Formate (HCOONa). In various embodiments of the present disclosure, the produced Hydrogen (H₂) can be used to support an energy imbalance market (EIM) and/or to provide a supplemental backup process to other Hydrogen (H₂) production mechanisms.

Production of hydrogen carriers via thermal decomposition of sodium salts are as described in Applicant's U.S. Patent Application Ser. No. 63/625,284 entitled “Thermal Decomposition of Sodium Formate for Direct In-Situ Methanol Production,” filed on Jan. 26, 2024, and U.S. Patent Application Ser. No. 63/507,057 entitled “Nuclear Reactor Integrated Energy Systems for the Direct Capture of Carbon Dioxide from Emissions Sources for Methanol Production,” filed on Jun. 8, 2023, each of which is incorporated herein by reference in their entireties.

Various embodiments of the present disclosure are directed to thermal and hydrothermal decomposition of inert sodium salts for hydrogen production. Inert sodium salts, such as sodium formate, can be transported easily and safely in large quantities and over long distances (e.g., via trucks or railcars). Additional processes, such as liquification and compression processes, and/or resources, such as cryogenic tankers, are not required to transport inert sodium salts as hydrogen carriers, thereby avoiding additional energy usage and/or greenhouse gas production compared to conventional hydrogen transportation.

When the inert sodium salt is heated to >360° C., they decompose to give off hydrogen. This process can be described as “thermal decomposition.” Thermal decomposition or thermolysis is a standard method or practice to produce different chemicals by applying thermal energy. The reaction is endothermic because thermal energy (heat) is applied to the materials to break the chemical bonds.

Various embodiments of the present disclosure are directed to hydrothermal decomposition of inert sodium salts. The use of hydrothermal decomposition to produce hydrogen is extremely efficient. The difference between the typical electrolysis technique and the hydrothermal decomposition is that electrolysis process depends on surface areas (stacks) while the hydrothermal decomposition is a volumetric reaction. Accordingly, hydrothermal decomposition is advantageous for hydrogen production and transportation as hydrothermal decomposition enables cheaper production of hydrogen as well as easier and safer transportation of reactants for hydrogen production.

In this disclosure, hydrothermal decomposition refers to a process involving thermally heating chemical(s) with super-heated steam to produce different chemicals. Super-heated steam, in various examples, refers to steam with a temperature above 450° C. In various embodiments of the present disclosure, super-heated steam can be generated from one or more nuclear reactors. The temperature of the steam generated by the nuclear reactor(s) can be between 300° C. to 800° C. Steam from the nuclear reactor(s) can be augmented, for instance, by using heaters and/or compressors, to produce super-heated steam with temperature >500° C. See NuScale Technical Report, Preliminary Assessment of NuScale Steam Production Rates for Industrial Applications, WP-139434 Rev3, May 2023. The super-heated steam reacts with the chemical compounds to generate materials that are not produced under the typical thermal decomposition process. For example, super-heated steam generated from nuclear reactor(s) can decompose the inert sodium salts to generate a large quantity of hydrogen that cannot typically be attained under the traditional thermal decomposition process.

Various embodiments of the present disclosure are directed to an integrated small modular nuclear reactor (SMR) system that can be emplaced as a baseload energy generator to produce electricity for the power grid during periods where energy production demand is high (“peak times”). During the period when the demand for electricity is low or below the typical baseload supply (“off-peak times”), then some of the reactor systems (e.g., SMRs) can be utilized to provide electricity and steam to high-temperature steam electrolysis cell (HTSE) stacks to produce Hydrogen (H₂) and Oxygen (O₂) for storage. The stored Hydrogen (H₂) and Oxygen (O₂) can be fed into an electrochemical device called a hydrogen fuel cell to generate electricity to support an EIM process. In some instances, the EIM time slot may typically be defined between 6:00 p.m. to 10:00 p.m. (about a 4-hour period).

In various embodiments, the SMR system of the present disclosure can be a permanent installation built at or near (e.g., roughly 1 km from) the location of an industrial process facility or can be a mobile or partially mobile system that is moved to and assembled at or near the location of the industrial process facility. More generally, the SMR system can be local (e.g., positioned at or near) to the industrial processes/operations it supports. For example, the SMR system can be located within 0.4 km (0.25 mile), within 0.8 km (0.5 mile), within 3.22 km (2 miles), within 4.82 km (3 miles), within 8.1 km (5 miles), or within more than 8.1 km of the industrial processes/operations it supports. In various embodiments, the SMR system is configured to supply a portion of electricity to a power grid.

Three pathways for Hydrogen (H₂) production from Sodium Formate (HCOONa) are shown below:

### Pathway 1—Hydrothermal Decomposition (Wet Process Utilizing Super-Heated Steam)

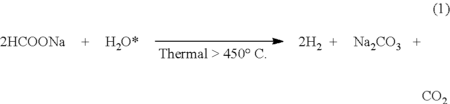

* super-heated steam (e.g., process steam from the SMR system)

When super-heated steam from a nuclear reactor system (e.g., an SMR system) is allowed to be injected into the system, the super-heated steam will react with Sodium Formate (HCOONa) and Hydrogen (H₂) is released and Carbon Dioxide (CO₂) is formed.

### Pathway 2—Thermal and Hydrothermal Decomposition (Combination Dry and Wet Process)

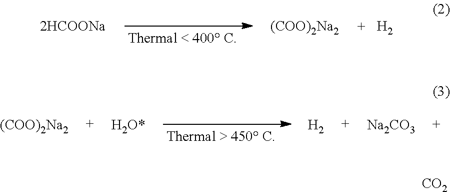

* super-heated steam (e.g., process steam) from the SMR system

A first dry process with a temperature around 360° C. causes the decomposition of Sodium Formate (HCOONa) to Sodium Oxalate ((COO)₂Na₂) and Hydrogen (H₂) gas. A second hydrothermal process using super-heated steam from a nuclear reactor system causes the reaction of Sodium Oxalate ((COO)₂Na₂) with the super-heated steam to form Carbon Dioxide (CO₂) gas, Sodium Carbonate (Na₂CO₃), and Hydrogen (H₂) gas.

In various embodiments of the present disclosure, the second hydrothermal process may be replaced with a thermal process (e.g., a dry process) as illustrated in Equations (4) and (5).

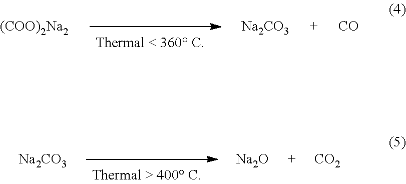

In various embodiments, the process includes a nuclear power plant providing power to a reverse osmosis (RO) operation to produce good quality water from seawater. The brine (NaCl solution) that is generated from the RO process is not released back into the ocean but is instead treated via the chlor-alkali membrane process to generate a carbon dioxide (CO₂) capturing solution containing sodium hydroxide (NaOH).

The NaOH solution is an intermediate material for the following specific applications: 1) carbon capture—to remove carbon dioxide (CO₂) from industrial sources and from the atmosphere via a Direct Air Capture (DAC) process; and 2) as an initiator to generate inert sodium salts, such as sodium formate, which is a Hydrogen carrier. Specifically, when an NaOH solution reacts with CO₂, Sodium Carbonates (Na₂CO₃) and Sodium Bicarbonates (NaHCO₃) are formed. Both Sodium Carbonates and Sodium Bicarbonates can react with simple carboxylic acids (e.g., formic acid) to produce an inert sodium salt (e.g., sodium formate).

The Detailed Description is set forth with reference to the accompanying figures. In the figures, the left-most digit(s) of a reference number identifies the figure in which the reference number first appears. The use of the same reference numbers in different figures indicates similar or identical items. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the same components on a larger scale or differently shaped for the sake of clarity.

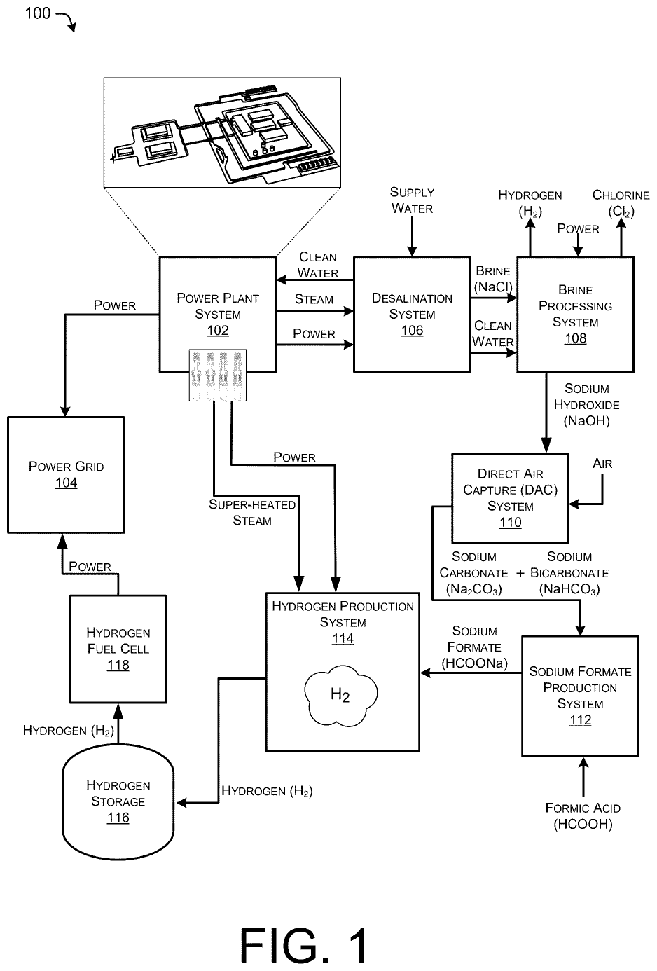

FIG. 1 schematically illustrates a representation of an integrated energy system 100 that includes a small modular nuclear reactor (SMR) system integrated with a Hydrogen (H₂) production system and an electrochemical device, such as a Hydrogen Fuel Cell. In various examples, the integrated energy system 100 includes a power plant system 102, a power grid 104, a desalination system 106, a brine processing system 108, a direct air capture (DAC) system 110, a Sodium Formate (HCOONa) production system 112, a Hydrogen (H₂) production system 114, a Hydrogen (H₂) storage 116, and a Hydrogen Fuel Cell 118. In various examples, one or more of the components of the integrated energy system 100 may be excluded or replaced with an equivalent component.

In various embodiments, the power plant system 102 may be configured to provide electrical power directly to the power grid 104. In various examples, the power plant system 102 may produce and deliver electrical power to the power grid 104. In some examples, the power plant system 102 may be configured to produce and provide the energy necessary for the power grid 104 to meet the consumer demand (e.g., higher demand during peak times, lower demand during off-peak times, etc.). The power plant system 102 may be configured to provide energy directly to the power grid 104 to meet energy demand due to other factors, such as, for instance, when an energy-producing plant that provides energy to the power grid 104 is unable to provide energy.

In some embodiments, the power plant system 102 may generate steam as a byproduct of the energy production process. In various instances, the power plant system 102 may be configured to provide steam and power to the desalination system 106. In various examples, the desalination system 106 may be configured to utilize the steam and power from the power plant system 102 to convert supply water into a concentrated NaCl solution (“brine”), clean water, and Carbon Dioxide (CO₂). The brine and the clean water may be directed into the brine processing system 108 and the Carbon Dioxide (CO₂) may be directed to the DAC system 110. The brine processing system 108 may be configured to convert brine into Sodium Hydroxide (NaOH), Hydrogen (H₂), and Chlorine (Cl₂). In some examples, the Sodium Hydroxide (NaOH) may be directed from the brine processing system 108 to the DAC system 110.

In contrast to conventional technology, the integrated energy system 100 may be utilized to capture Carbon Dioxide (CO₂) without input of any natural gas. For example, the DAC system 110 may utilize a DAC process to capture Carbon Dioxide (CO₂) by pulling in atmospheric air, then through a series of chemical reactions, utilizing steam from the SMR system to extract Carbon Dioxide (CO₂), while the rest of the air may be returned to the atmosphere. The DAC process can start with an air contactor—a large structure modelled after industrial cooling towers. A large fan can pull air into this structure, where it passes over thin plastic surfaces that have sodium hydroxide solution flowing over them. This non-toxic solution may chemically bind with the CO₂ molecules, thereby removing the Carbon Dioxide (CO₂) molecules from the air and trapping them in the liquid solution as a carbonate salt.

In some examples, the air contactor can begin the DAC process by drawing in air from the atmosphere to an air contactor where the air may pass over plastic surfaces that have a Sodium Hydroxide (NaOH) solution, a carbon-dioxide capture solution, flowing over them. The Carbon Dioxide (CO₂) molecules in the air may bind with the Sodium Hydroxide (NaOH) to create Sodium Carbonate (Na₂CO₃) and water, as expressed by Equation 6, shown below:

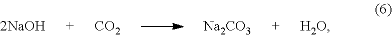

where 2NaOH is sodium hydroxide (the carbon-dioxide capture solution), CO₂ is carbon dioxide within the air, Na₂CO₃ is the sodium carbonate created when the airborne CO₂ molecules bind with the sodium hydroxide capture solution, and H₂O is created, along with the sodium carbonate, when the airborne CO₂ molecules bind with the NaOH capture solution. The Na₂CO₃ solution can react with additional CO₂ gas to form Sodium Bicarbonate (NaHCO₃) solution, as expressed by Equation 7, shown below:

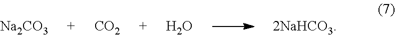

In various embodiments, the DAC system 110 may be configured to convert the Sodium Hydroxide (NaOH) from the brine processing system 108, the isolated Carbon Dioxide (CO₂), and air into Sodium Carbonate (Na₂CO₃) and Sodium Bicarbonate (NaHCO₃). In some examples, the DAC system 110 may receive Carbon Dioxide (CO₂) from the desalination system 106. In some examples, the Sodium Carbonate (Na₂CO₃) and Sodium Bicarbonate (NaHCO₃) from the DAC system 110 may be directed into the Sodium Formate (HCOONa) production system 112.

The Sodium Formate (HCOONa) production system 112 is configured to generate Sodium Formate (HCOONa). For instance, Sodium Formate (HCOONa) may be produced by neutralizing Formic Acid (HCOOH) with Sodium Hydroxide (NaOH). In some embodiments, Sodium Formate (HCOONa) may be produced in a large-scale inexpensively from Formic Acid (HCOOH) via carbonylation of Methanol (CH₃OH) followed by adding water to the resulting Methyl Formate (HCOOCH₃). In some cases, the Sodium Formate (HCOONa) production system 112 may utilize the Sodium Carbonate (Na₂CO₃) and Sodium Bicarbonate (NaHCO₃) to generate Sodium Formate (HCOONa). In various embodiments, the Sodium Formate (HCOONa) production system 112 may be configured to receive Sodium Formate (HCOONa) from an external source.

The Sodium Formate (HCOONa) from the Sodium Formate (HCOONa) production system 112 may be directed into the Hydrogen (H₂) production system 114. In some examples, the Hydrogen (H₂) production system 114 may be configured to receive steam from the power plant system 102 and Sodium Formate (HCOONa) from the Sodium Formate (HCOONa) production system 112 to produce Hydrogen (H₂).

In various embodiments, the proximity between the power plant system 102 and the Hydrogen (H₂) production system 114 enables the use of the steam from the power plant system 102 for Hydrogen (H₂) production. In some examples, the power plant system 102 includes one or more nuclear reactors configured to generate electrical power and, as a byproduct, the steam. However, nuclear reactors are conventionally located at considerable distances from populated areas and from other chemical processing facilities due to safety considerations as well as public concerns. In various embodiments of the present disclosure, the use of small modular nuclear reactors can enable the power plant system 102 to be located within a smaller distance of the Hydrogen (H₂) production system 114. Accordingly, the steam from the power plant system 102 can be effectively transported to the Hydrogen (H₂) production system 114 and other facilities in order to, for instance, facilitate one or more thermal and/or hydrothermal processes by the Hydrogen (H₂) production system 114.

The steam may be used to facilitate hydrothermal and/or thermal processes for the production of Hydrogen (H₂). For instance, the steam may be used to maintain an internal temperature of the Hydrogen (H₂) production system 114 to facilitate one or more thermal processes (e.g., dry processes). In some embodiments, the steam is introduced into the Hydrogen (H₂) production system 114 to of Sodium Formate (HCOONa) to facilitate one or more hydrothermal processes (e.g., wet processes). Hydrothermal processes, according to various embodiments of the present disclosure, can enable production of higher quantities of Hydrogen (H₂) than conventional thermal processes. In some examples, the steam from the power plant system 102 may be augmented to generate super-heated steam (e.g., steam with a temperature above 450° C.). In various embodiments, the electrical power generated by the power plant system 102 may be used to augment the steam from the power plant system 102. In some cases, a thermal recovery system that is powered by the power grid 104 may be configured to augment the steam from the power plant system 102.

In various embodiments, the Hydrogen (H₂) production system 114 includes one or more reaction chambers. For instance, the Hydrogen (H₂) production system 114 may include a first reaction chamber with an internal temperature maintained within a first range using the steam from the power plant system 102. In some examples, the Hydrogen (H₂) production system 114 may include a second reaction chamber with an internal temperature with a second range using the steam from the power plant system 102.

In some cases, the first reaction chamber is configured to receive steam from the power plant system 102 into the first reaction chamber (e.g., into an internal cavity of the first reaction chamber) to facilitate hydrothermal decomposition of Sodium Formate (HCOONa) as represented by Equation (1):

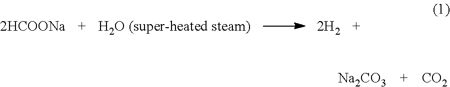

The steam may be used to maintain an internal temperature of the first reaction chamber in a range of 450° C. to 800° C. In some embodiments, the steam may be used along with a different thermal energy source (e.g., a heater) may be used to maintain the internal temperature of the first reaction chamber.

In some cases, the first reaction chamber is configured to receive steam from the power plant system 102 onto an external surface (e.g., a surface, a jacket, an insulative layer, etc.) of the first reaction chamber to facilitate thermal decomposition of Sodium Formate (HCOONa) as represented by Equation (2):

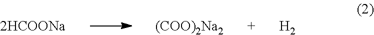

The steam may be used to maintain an internal temperature of the first reaction chamber in a range of 300° C. to 400° C. In some embodiments, the steam and/or a different thermal energy source (e.g., a heater) may be used to maintain the internal temperature of the first reaction chamber.

In various instances, the Sodium Oxalate ((COO)₂Na₂) generated in the first reaction chamber is introduced into a second reaction chamber of the Hydrogen (H₂) production system 114. The Sodium Oxalate ((COO)₂Na₂) may be hydrothermally decomposed in the second reaction chamber by introducing the steam from the power plant system 102 into the second reaction chamber, as represented by Equation (3):

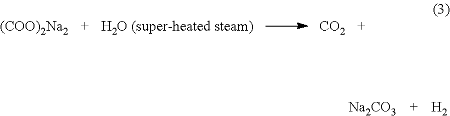

The steam may be used to maintain an internal temperature of the second reaction chamber in a range of 450° C. to 800° C. In some embodiments, the steam may be used along with a different thermal energy source (e.g., a heater) may be used to maintain the internal temperature of the second reaction chamber.

In various examples, the Hydrogen (H₂) produced by the Hydrogen (H₂) production system 114 may be stored in the Hydrogen (H₂) storage 116. In some cases, the Hydrogen (H₂) is directed from the Hydrogen (H₂) storage 116 to the Hydrogen Fuel Cell 118. The Hydrogen Fuel Cell 118 may be configured to utilize the Hydrogen (H₂) from the Hydrogen (H₂) storage 116 to produce electrical power. The Hydrogen Fuel Cell 118 may be a Proton Exchange Membrane (PEM) Fuel Cell, a Solid Oxide Fuel Cell (SOFC), or a Liquid Carbonate Fuel Cell (LCFC). In various embodiments, the electrical power produced by the Hydrogen Fuel Cell 118 may be directed to the power grid 104.

It is understood that the integrated energy system 100 may be configured such that the power plant system 102 may simultaneously produce electrical power directly to the power grid 104 and produce Hydrogen (H₂) via the desalination system 106, the brine processing system 108, the direct air capture (DAC) system 110, the Sodium Formate (HCOONa) production system 112, and the Hydrogen (H₂) production system 114. It is also understood that the integrated energy system 100 may be configured such that the power plant system 102 may simultaneously produce and provide electrical power directly to the power grid 104 while the Hydrogen Fuel Cell 118 is utilizing Hydrogen (H₂) from the Hydrogen (H₂) storage 116 to produce and provide electrical power directly to the power grid 104.

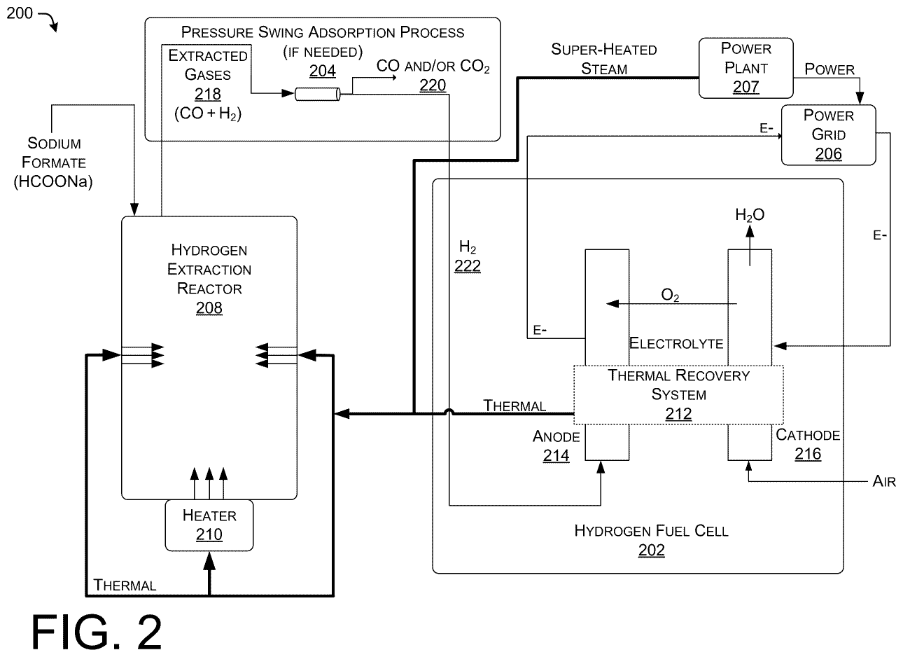

FIG. 2 illustrates a steady-state Hydrogen (H₂) production process 200 (“process 200”) utilizing Sodium Formate (HCOONa) and a Hydrogen Fuel Cell. In various embodiments, process 200 may include a Hydrogen Fuel Cell 202, a pressure swing adsorption process 204, a power grid 206, a power plant 207, and a Hydrogen (H₂) extraction reactor 208. The Hydrogen (H₂) extraction reactor 208 may include a Hydrogen (H₂) extraction reactor heater 210. The Hydrogen Fuel Cell 202, in some cases, may include the anode 214 and the cathode 216.

In some embodiments, Sodium Formate (HCOONa) is fed into the Hydrogen (H₂) extraction reactor 208. The Hydrogen (H₂) extraction reactor heater and the Hydrogen Fuel Cell 202 may receive heat from the thermal recovery system 212 that is powered by the power grid 206 to keep the process 200 at operational temperatures. Hydrogen (H₂) may also be injected from an external source (e.g., a tanker truck, a storage tank, etc.) into the Hydrogen Fuel Cell 202 to generate electricity and reduce energy from the power grid 206.

In various embodiments, the Hydrogen (H₂) extraction reactor 208 may receive Sodium Formate (HCOONa). It is understood that the Hydrogen (H₂) extraction reactor 208 may receive Sodium Formate (HCOONa) in a solid-state form or as a powder. In some examples, the Hydrogen (H₂) extraction reactor 208 may be maintained with an internal temperature higher than 450° C. For example, the Hydrogen (H₂) extraction reactor 208 includes one reactor with an internal temperature between 450° C. to 800° C. In various embodiments, the Hydrogen (H₂) extraction reactor 208 may be maintained with an internal temperature lower than 450° C. For instance, the Hydrogen (H₂) extraction reactor 208 may include a first reaction chamber with an internal temperature between 300° C. to 400° C. and a second reaction chamber with an internal temperature between 450° C. to 800° C.

In various embodiments, the power grid 206 is powered by the power plant 207 (e.g., the power plant system 102 described with respect to FIG. 1). The power plant 207 includes one or more nuclear reactors, such as small modular nuclear reactors, configured to produced steam. For instance, the steam may be collected as a by-product of the energy production process of the power plant 207. In some examples, the steam may be used to maintain temperature in the Hydrogen (H₂) extraction reactor 208. In some examples, the steam may be introduced into the Hydrogen (H₂) extraction reactor 208 to provide thermal energy and water.

In some embodiments, the Hydrogen (H₂) extraction reactor heater 210 may utilize electricity from the power grid 206 to maintain the Hydrogen (H₂) extraction reactor internal temperature. In some embodiments, the Hydrogen (H₂) extraction reactor heater 210 may utilize thermal energy recovered from the thermal recovery system 212 to maintain the Hydrogen (H₂) extraction reactor internal temperature.

During its operation, the Hydrogen (H₂) extraction reactor 208 may process Sodium Formate (HCOONa) to produce extracted gases 218 (e.g., Carbon Monoxide (CO), Carbon Dioxide (CO₂), er and Hydrogen (H₂)). It is understood that the extracted gases 218 may be a mixture of gases with varied concentrations. The extracted gases 218 may be directed to the Pressure Swing Adsorption (PSA) process 204 to be separated into separate gases (e.g., a mixture of Hydrogen (H₂) and Carbon Dioxide (CO₂))

In various embodiments, the Hydrogen (H₂) 222 may be directed to the Hydrogen Fuel Cell 202. It is understood the process 200 may use a Hydrogen (H₂) fuel cell other than the type depicted within FIG. 2. The Hydrogen (H₂) 222 may be directed to the anode 214 of the Hydrogen Fuel Cell 202 to be oxidized and separated into electrons and positively charged hydrogen ions. The electrons may be directed to the power grid 206 to generate electricity before being directed to the cathode 216. In some examples, air (e.g., atmospheric air containing Oxygen (O₂)) may be directed into the cathode 216 of the Hydrogen Fuel Cell 202. The Oxygen (O₂) in the cathode 216 may combine with the electrons directed into in the cathode 216 from the power grid 206 and the positively charged hydrogen particles that traveled from the anode 214 to the cathode 216 via the electrolyte to generate water (H₂O). The oxidation of the Hydrogen (H₂) 222 and generation of water (H₂O) in the Hydrogen Fuel Cell 202 produces heat. In various instances, the thermal recovery system 212 captures the heat generated by the Hydrogen Fuel Cell 202 and direct the heat to Hydrogen (H₂) extraction reactor 208.

It is understood that the thermal recovery system 212 may be a system utilizing a thermal fluid to transfer heat, a system utilizing a Stirling engine with electrical component to power a heater, or any other system suitable to recover and reuse the heat generated by the Hydrogen Fuel Cell 202.

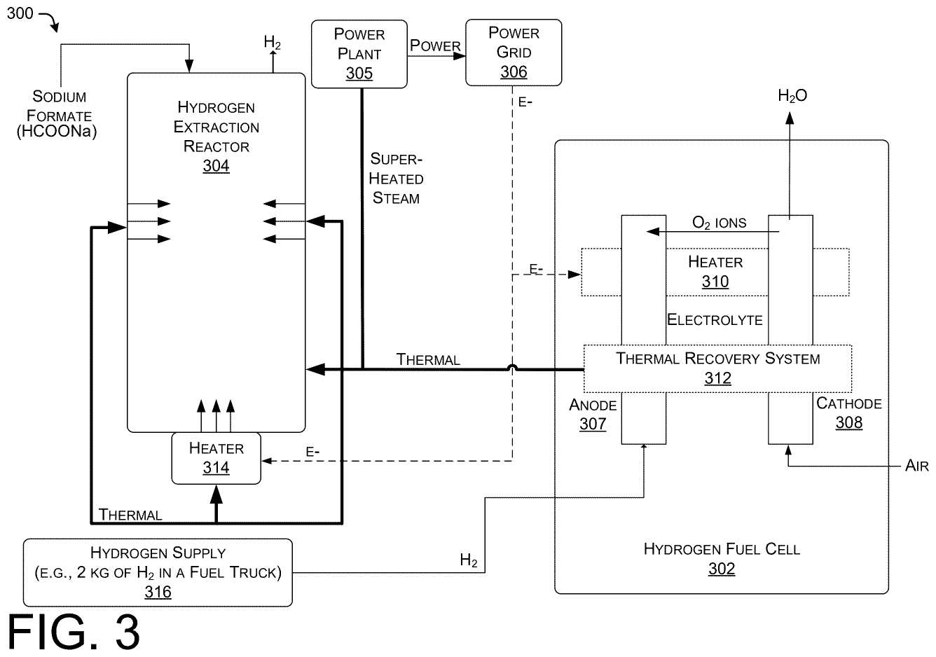

FIG. 3 illustrates a steady-state Hydrogen (H₂) production process 300 (“process 300”) utilizing Sodium Formate (HCOONa) and a Hydrogen Fuel Cell 302. In various embodiments, the process 300 may include a Hydrogen Fuel Cell 302, a Hydrogen (H₂) extraction reactor 304, a power plant 305, and a power grid 306. The Hydrogen Fuel Cell 302 may include an anode 307, a cathode 308, a heater 310, and a thermal recovery system 312. The Hydrogen (H₂) extraction reactor 304, in various examples, may include a Hydrogen (H₂) extraction reactor heater 314. In some cases, the anode 307 may be configured to receive Hydrogen (H₂) from a Hydrogen (H₂) supply 316. It is understood that the Hydrogen (H₂) supply 316 may include a Hydrogen (H₂) production system, a permanent Hydrogen (H₂) storage tank, and/or a temporary Hydrogen (H₂) storage tank (e.g., a Hydrogen (H₂) fuel truck, portable tanks(s), etc.). In an embodiment, the Hydrogen Fuel Cell 302 may only need approximately 2 kg of Hydrogen (H₂) from an external source during startup. It is understood that in some embodiments, external Hydrogen (H₂) may only be required for the Hydrogen Fuel Cell 302 during startup, and that after startup, the Hydrogen Fuel Cell 302 may receive necessary Hydrogen (H₂) from the Hydrogen (H₂) extraction reactor 304.

In various embodiments, the power plant 305 provides thermal energy via steam to the Hydrogen (H₂) extraction reactor 304. The steam may be used to maintain an internal temperature of the Hydrogen (H₂) extraction reactor 304. For instance, the steam may be used to maintain an internal temperature of the Hydrogen (H₂) extraction reactor 304 between 300° C. to 360° C. In some examples, the steam may be used to maintain an internal temperature between of the Hydrogen (H₂) extraction reactor 304 between 450° C. to 800° C. In various embodiments, the steam may be introduced into the Hydrogen (H₂) extraction reactor 304 to facilitate hydrothermal decomposition of Sodium Formate (HCOONa). In various embodiments, the Hydrogen (H₂) extraction reactor 304 is configured to use the steam to maintain an internal temperature without introducing steam into the Hydrogen (H₂) extraction reactor 304. Accordingly, the steam can be used to facilitate thermal decomposition (e.g., a dry process) of Sodium Formate (HCOONa).

In some examples, the power grid 306 may provide electrical power to the heater 310 and the Hydrogen (H₂) extraction reactor heater 314. In various cases, the heater 310 and the Hydrogen (H₂) extraction reactor heater 314 may only require the use of electrical power from the power grid 306 during startup. In some embodiments, electrical power may only be required for the heater 310 during startup, and that after startup, the Hydrogen Fuel Cell 302 may no longer require the use of the heater 310. For example, in various instances, the Hydrogen Fuel Cell 302 may use the heater 310 during startup because the normal steady-state operation of the Hydrogen Fuel Cell 302 may generate the heat necessary to sustain the operation of the Hydrogen Fuel Cell 302.

In some embodiments, electrical power may only be required for the Hydrogen (H₂) extraction reactor heater 314 during startup, and that after startup, the Hydrogen (H₂) extraction reactor 304 may no longer require the use of the Hydrogen (H₂) extraction reactor heater 314. For example, the Hydrogen (H₂) extraction reactor heater 314 may use electrical energy to provide the heat necessary for the operation of the Hydrogen (H₂) extraction reactor 304 during startup. In various cases, the normal steady-state operation of the Hydrogen Fuel Cell 302 may generate heat, which may be recovered by the thermal recovery system 312 and transferred to the Hydrogen (H₂) extraction reactor 304. During normal steady-state operation, the heat recovered by the thermal recovery system 312 may be adequate for operation of the Hydrogen (H₂) extraction reactor 304. In various cases, the steam generated by the power plant 305 is sufficient for operation of the Hydrogen (H₂) extraction reactor 304.

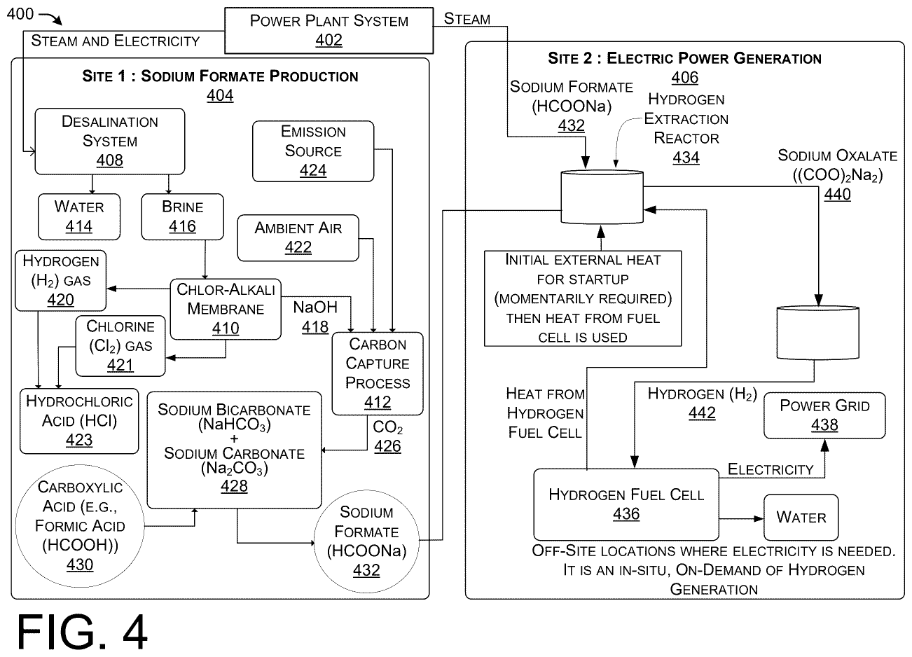

FIG. 4 illustrates in-situ and on-demand Hydrogen (H₂) production system (“system 400”) to support emergency and limited energy imbalance market (EIM) using Sodium Formate (HCOONa). In various embodiments, the system 400 may include a power plant system 402, a first site 404, and a second site 406. The first site 404 may be used for Sodium Formate (HCOONa) production. The second site 406 may be an off-site location (i.e., located wherever electricity is needed that may not be in proximity to the power plant system 400). The second site 406 may provide in-situ on-demand Hydrogen (H₂) generation. In various cases, the power plant system 400 may include a SMR system.

In some examples, the first site 404 may be used for Sodium Formate (HCOONa) production. The first site 404 may include the desalination system 408, the chlor-alkali membrane 410, and the carbon capture process 412. It is understood the chlor-alkali membrane 410 may include any type of electrolysis system and/or process configured to process brine into Sodium Hydroxide (NaOH). At the first site 404, the power plant system 402 may supply steam and electricity to the desalination system 408. The desalination system 408 may produce water 414 and brine 416 (i.e., a concentrated Sodium Chloride (NaCl) solution). The brine 416 may be directed into the chlor-alkali membrane 410. The chlor-alkali membrane 410 may be configured to receive the brine 416 and generate Sodium Hydroxide (NaOH) 418, Hydrogen (H₂) gas 420, and Chlorine (Cl₂) gas 421 via electrolysis. In some cases, the Hydrogen (H₂) gas 420 and the Chlorine (Cl₂) gas 421 may be combined to form Hydrochloric Acid (HCl) 423. The production of Hydrochloric Acid (HCl) may be represented by the equation below:

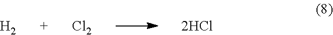

In some examples, the carbon capture process 412 may receive ambient air 422 (e.g., atmospheric air containing Carbon Dioxide (CO₂)) and/or an emission source 424 (e.g., gases containing Carbon Dioxide (CO₂) released as an emission from a process, machine, device, etc.) and produce Carbon Dioxide (CO₂) 426, which may be useful for industrial processes. In various cases, the Sodium Hydroxide (NaOH) 418 may be combined with the Carbon Dioxide (CO₂) 426 to generate Sodium Bicarbonate (NaHCO₃) and Sodium Carbonate (Na₂CO₃) 428. In various cases, a carboxylic acid (e.g., Formic Acid (HCOOH)) 430 may be reacted with the Sodium Bicarbonate (NaHCO₃) and Sodium Carbonate (Na₂CO₃) 428 to produce Sodium Formate (HCOONa) 432 that may be transported to the second site 406.

The second site 406 may include the Hydrogen (H₂) extraction reactor 434, an electrochemical device (e.g., Hydrogen Fuel Cell 436), and the power grid 438. In some instances, the Hydrogen (H₂) extraction reactor 434 receives steam (e.g., super-heated steam) from the power plant system 402. While FIG. 4 illustrates the power plant system 402 providing steam to a first reaction chamber of the Hydrogen (H₂) extraction reactor 434, embodiments of the present disclosure are not so limited. In various embodiments, the power plant system 402 provides steam to additional reaction chamber of the Hydrogen (H₂) extraction reactor 434. The steam may be used to maintain an internal temperature of one or more reaction chambers of the Hydrogen (H₂) extraction reactor 434. In some cases, a first reaction chamber of the Hydrogen (H₂) extraction reactor 434 may receive Sodium Formate (HCOONa) 432 to produce Sodium Oxalate ((COO)₂Na₂) 440 and Hydrogen (H₂) 442. The first reaction chamber may have an internal temperature within a range of 300° C. to 400° C. The conversion of the Sodium Formate (HCOONa) 432 to Sodium Oxalate ((COO)₂Na₂) 440 and Hydrogen (H₂) 442 may be represented by the reaction below:

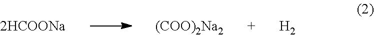

In various embodiments, the Sodium Oxalate ((COO)₂Na₂) 440 is directed into a second reaction chamber of the Hydrogen (H₂) extraction reactor 434. The second reaction chamber of the Hydrogen (H₂) extraction reactor 434 may be maintained at an internal temperature of 450° C. to 800° C. For instance, the power plant system 402 may provide steam into the second reaction chamber of the Hydrogen (H₂) extraction reactor 434 to facilitate the hydrothermal decomposition of Sodium Oxalate ((COO)₂Na₂) 440. The conversion of Sodium Oxalate ((COO)₂Na₂) 440 to Carbon Dioxide (CO₂), Sodium Carbonate (Na₂CO₃), and Hydrogen (H₂) 442 may be represented by the reaction below:

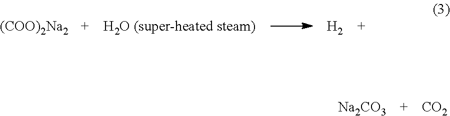

While FIG. 4 illustrates the first reaction chamber and the second reaction chamber of the Hydrogen (H₂) extraction reactor 434, embodiments of the present disclosure are not so limited. In various embodiments, the Hydrogen (H₂) extraction reactor 434 includes a single reaction chamber configured to receive super-heated steam from the power plant system 402. The single reaction chamber of the Hydrogen (H₂) extraction reactor 434 may maintain an internal temperature of at least 450° C. The steam, in various examples, facilitates the hydrothermal decomposition of Sodium Formate (HCOONa) to Hydrogen (H₂), Sodium Carbonate (Na₂CO₃), and Carbon Dioxide (CO₂), as represented by the reaction below:

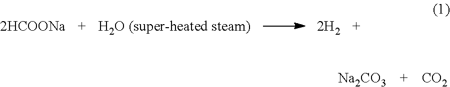

The Hydrogen (H₂) 442 may be directed to the Hydrogen Fuel Cell 436, which may be configured to convert the Hydrogen (H₂) 442 into electricity and water. It is understood that the Hydrogen (H₂) 442 may be directly directed to the Hydrogen Fuel Cell 436 and/or directed to a Hydrogen (H₂) tank (i.e., tanker truck, portable storage tank, permanently installed tank, etc.). The electricity produced by the Hydrogen Fuel Cell 436 may be directed to the power grid 438. In some embodiments, the Hydrogen Fuel Cell 436 may operate to produce electricity as needed to support an EIM. In various instances, the Hydrogen Fuel Cell 436 may generate heat during operation which may be directed to the Hydrogen (H₂) extraction reactor 434.

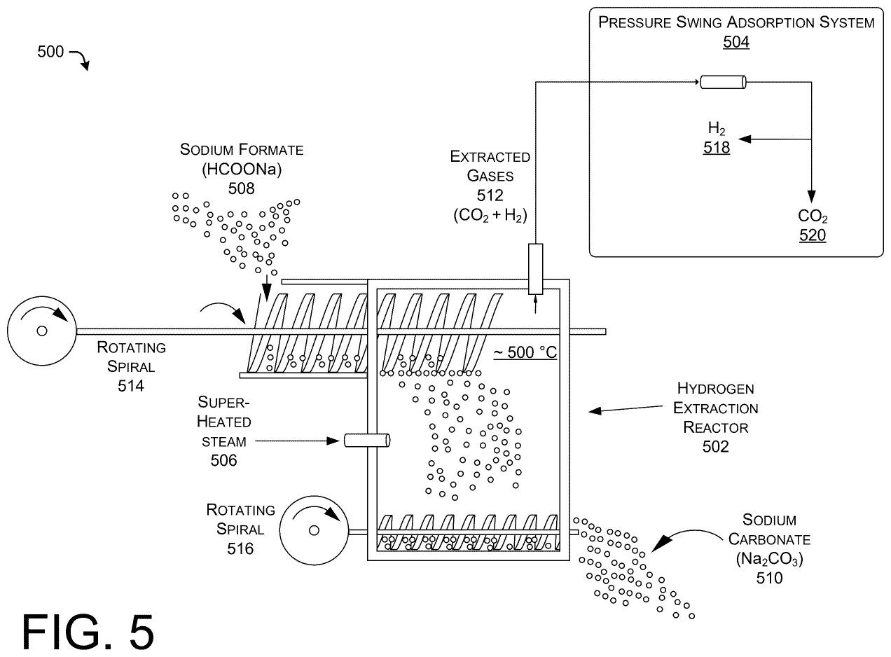

FIG. 5 schematically illustrates a representative schematic diagram of a Hydrogen (H₂) extraction reactor system 500 (“system 500”) during steady state operations for the hydrothermal decomposition of Sodium Formate (HCOONa) (i.e., wet process). In various embodiments, the system 500 may include a Hydrogen (H₂) extraction reactor 502 and a pressure swing adsorption system 504. The Hydrogen (H₂) extraction reactor 502 may be configured to receive super-heated steam 506 and Sodium Formate (HCOONa) 508 and produce Sodium Carbonate (Na₂CO₃) 510 and/or extracted gases 512. The Sodium Formate (HCOONa) 508 may be directed, via the rotating spiral 514 (e.g., first rotating spiral), into an upper portion of the Hydrogen (H₂) extraction reactor 502. The Hydrogen (H₂) extraction reactor 502 may have an internal temperature in a range of 450° C. to 800° C. In various examples, the Hydrogen (H₂) extraction reactor 502 may have an internal temperature in a range of ˜500° C. The super-heated steam 506 may be steam (e.g., process steam) from an SMR system.

In various cases, the rotating spiral 514 may be utilized to convert the Sodium Formate (HCOONa) 508 between particles of different sizes. For example, the rotating spiral 514 may be utilized to convert the Sodium Formate (HCOONa) 508 from relatively coarser (e.g., larger) particles to relatively finer (e.g., smaller) particles. The rotating spiral 514 may be utilized to assist in a conversion between the Sodium Formate (HCOONa) 508 to the Sodium Carbonate (Na₂CO₃) 510. The rotating spiral 514 may be utilized to maintain the temperature in the Hydrogen (H₂) extraction reactor 502 by providing a means to feed the Sodium Formate (HCOONa) 508 into the upper portion of the Hydrogen (H₂) extraction reactor 502 while minimizing the potential for heat loss. The rotating spiral 514 may be utilized to feed the Sodium Formate (HCOONa) 508 into the Hydrogen (H₂) extraction reactor 502 at a steady rate. The rotating spiral 514 may be a metal rotating spiral (e.g., an auger), which may be located partially and/or fully in the first Hydrogen (H₂) extraction reactor 502. In some embodiments, the rotating spiral 514 may be operated by a control system utilized to control any portion of the system 500 to rotate (e.g., spin) at one or more predetermined, and/or dynamically determined (e.g., in real-time) speeds during any operation of the system 500.

In an embodiment, the Sodium Formate (HCOONa) 508, may receive thermal energy as a result of the temperature inside the Hydrogen (H₂) extraction reactor 502. The internal temperature of the Hydrogen (H₂) extraction reactor 502 may cause the Sodium Formate (HCOONa) 508 to rapidly decompose into the extracted gases 512 and the Sodium Carbonate (Na₂CO₃) 510. In the embodiment, the extracted gases 512 may be produced instantaneously following the decomposition of the Sodium Formate (HCOONa) 508 into the Sodium Carbonate (Na₂CO₃) 510. In the embodiment, the resulting Sodium Carbonate (Na₂CO₃) 510 sinks to the bottom of the Hydrogen (H₂) extraction reactor 502 while still being thermally hot. The rotating spiral 516 (e.g., second rotating spiral) may transfer the thermally hot Sodium Carbonate (Na₂CO₃) 510 from the bottom of the Hydrogen (H₂) extraction reactor 502 (i.e., the lower portion of the Hydrogen (H₂) extraction reactor) to outside the Hydrogen (H₂) extraction reactor 502 for collection and/or additional industrial processing.

In various embodiments, the extracted gases 512 may include a mixture of Carbon Dioxide (CO₂) and Hydrogen (H₂). The pressure swing adsorption system 504 may be used to separate the extracted gases 512 into Hydrogen (H₂) 518 and Carbon Dioxide (CO₂) 520. In some examples, the Hydrogen (H₂) 518 and the Carbon Dioxide (CO₂) 520 may be used for future processing.

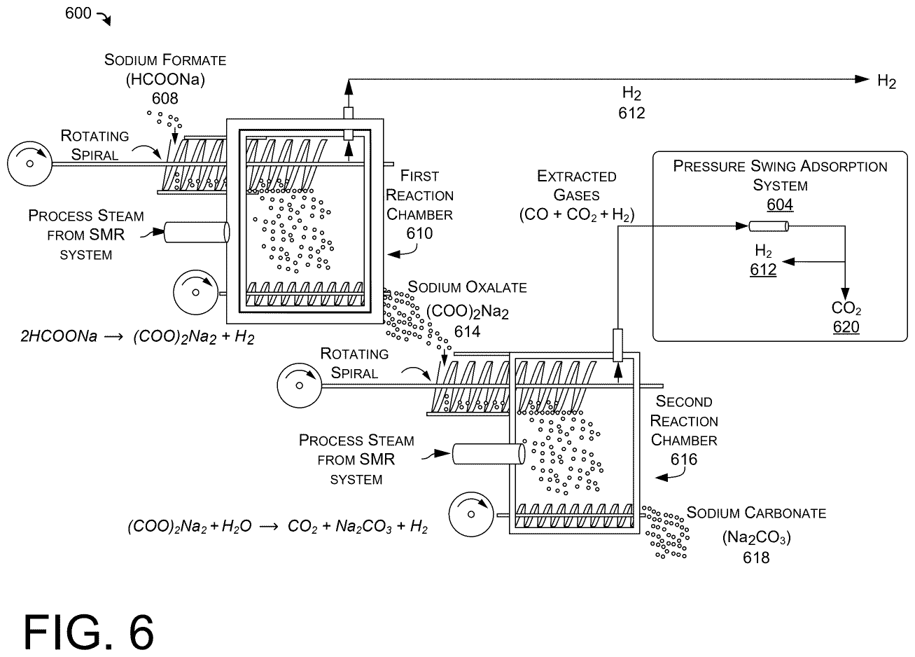

FIG. 6 schematically illustrates a representative schematic diagram of a syngas production system 600 of Hydrogen (H₂) from Sodium Formate (HCOONa). The system 600 may include a first thermal reaction chamber 610 utilized to output hydrogen gas 612 and/or sodium oxalate 614. The first thermal reaction chamber 610 may be configured to maintain an internal temperature of 300° C. to 400° C. In some embodiments, the first thermal reaction chamber 610 may be configured to maintain an internal temperature of 300° C. to 360° C. For instance, process steam (e.g., super-heated steam) from a small modular nuclear reactor (SMR) may be used to maintain the internal temperature of the first thermal reaction chamber 610. In an embodiment, the sodium formate 608 may be fed into the first thermal reaction chamber 610 utilizing an auger. Although depicted within FIG. 6 and described herein as an auger and a rotating spiral, various methods may be used to feed the sodium formate 608 into the first thermal reaction chamber 610.

In an embodiment, the first reaction chamber utilizes process steam from a Small Modular Nuclear Reactor (SMR) system to maintain the temperature within the first thermal reaction chamber 610 at a range of between 300° C. to 400° C. In an embodiment, maintaining the temperature of the first thermal reaction chamber 610 at a range of between 300° C. to 400° C. may cause the production of Hydrogen (H₂) 612 and Sodium Oxalate ((COO)₂Na₂) 614 from the Sodium Formate (HCOONa) 608, as demonstrated by Equation 2, shown below:

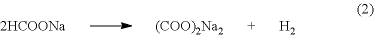

where 2HCOONa is sodium formate being introduced into the first reaction chamber, (COO)₂Na₂ is sodium oxalate produce by raising the temperature of the sodium formate, and H₂ is hydrogen gas produced, along with the sodium oxalate.

The thermal shock wave to the Sodium Formate (HCOONa) 608, as a result of the temperature inside the first thermal reaction chamber 610, may cause the Sodium Formate (HCOONa) 608 powder to rapidly decompose into Hydrogen (H₂) 612 and Sodium Oxalate ((COO)₂Na₂) 614. In the embodiment, the Hydrogen (H₂) 612 may be produced instantaneously following the decomposition of the Sodium Formate (HCOONa) 608. In the embodiment, the resulting Sodium Oxalate ((COO)₂Na₂) 614 may sink to the bottom of the first thermal reaction chamber 610 while still being thermally hot.

The system 600 may include a second thermal reaction chamber 616 utilized to output Carbon Dioxide (CO₂) 620, Sodium Carbonate (Na₂CO₃) 618, and/or Hydrogen (H₂) 612 via hydrothermal decomposition of the Sodium Oxalate ((COO)₂Na₂) 614. In some embodiments, the second thermal reaction chamber 616 can be utilized to output a combination of Carbon Monoxide (CO) and Sodium Carbonate (Na₂CO₃) and/or a combination of Carbon Monoxide (CO), Carbon Dioxide (CO₂) 620, and Sodium Oxide (Na₂O).

In an embodiment the Sodium Oxalate ((COO)₂Na₂) 614 may be transferred to the second reaction chamber 616. In an embodiment, the Sodium Oxalate ((COO)₂Na₂) 614 may be directly fed into the second reaction chamber 616 utilizing an auger. Although depicted within FIG. 6 and described herein as an auger and a rotating spiral, various methods may be used to feed the Sodium Oxalate ((COO)₂Na₂) 614 into the second reaction chamber 616.

In an embodiment the second reaction chamber 616 utilizes process steam from the SMR system and/or compressed heating to maintain a temperature within the second reaction chamber 616 of at least 500° C. Process steam from a small modular nuclear reactor (SMR) may be introduced into the second thermal reaction chamber 616, causing a reaction between water (e.g., from the super-heated steam) and Sodium Oxalate ((COO)₂Na₂) 614. In an embodiment, maintaining the temperature of the second reaction chamber 616 at a temperature of at least 500° C. causes hydrothermal decomposition of the Sodium Oxalate ((COO)₂Na₂) 614 into Sodium Carbonate (Na₂CO₃) 618, Carbon Dioxide (CO₂) 620, and Hydrogen (H₂) 612 as illustrated by the following Equation (3):

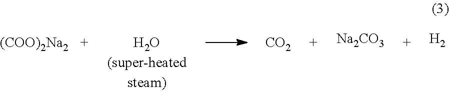

In some examples, the hydrothermal process described with reference to the second reaction chamber 616 can be replaced by a thermal process (e.g., dry process) as illustrated by the following equation:

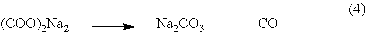

In some examples, the SMR system, including all support systems (e.g., electrical production system, steam transmission system, energy integration system, water treatment system, chemical production system, DAC system, gasification system, syngas production system, etc.) operably coupled thereto, may be physically located on a singular site having a threshold perimeter. In various cases, each system and or support system may be located less than a threshold distance (e.g., roughly 1 km) of the SMR system. In some cases, each support system can be a permanent, at or near (e.g., roughly within 1 km) of the SMR. In various embodiments, each system and support system may be assembled and/or constructed within a threshold radius of the epicenter of the SMR system site.

In some examples, aspects of the present technology may be directed generally toward IESs, such as for use in green industrial processes that produce few or no carbon emissions, and associated devices and methods. The industrial processes can include, for example, Carbon Dioxide (CO₂) production.

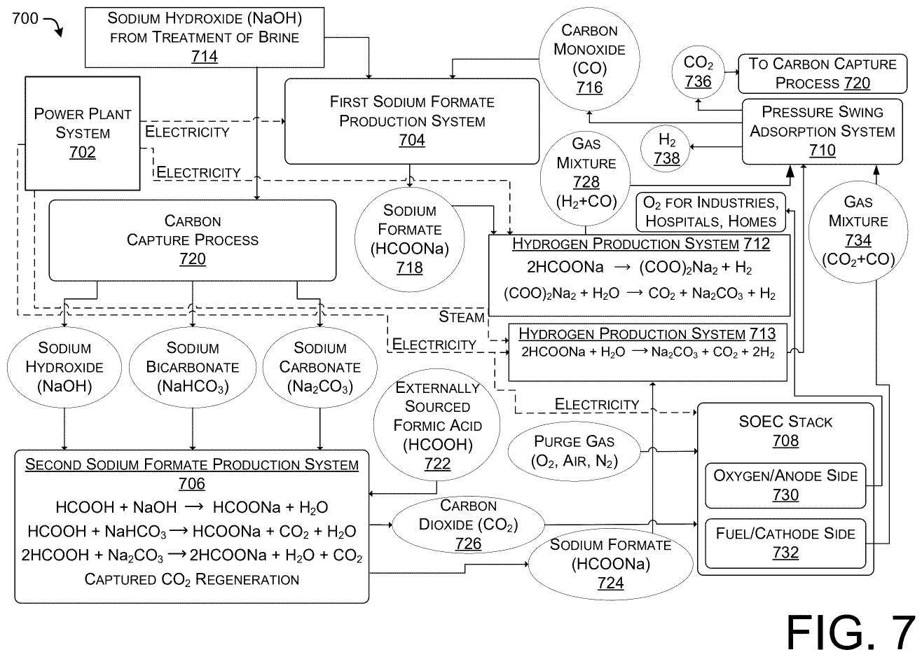

FIG. 7 illustrates Hydrogen (H₂) production process 700 (“process 700”) that uses multiple Sodium Formate (HCOONa) production systems simultaneously. The process 700 may include a power plant system 702, a first Sodium Formate (HCOONa) production system 704, a second Sodium Formate (HCOONa) production system 706, an electrochemical device (e.g., solid oxide electrolysis cell (SOEC) stack 708), a pressure swing adsorption system 710, a Hydrogen (H₂) production system 712, and a Hydrogen (H₂) production system 713. In some embodiments, the power plant system 702 may provide electricity to the first Sodium Formate (HCOONa) production system 704, the Hydrogen (H₂) production system 712, the Hydrogen (H₂) production system 713, and the SOEC stack 708. In some examples, the power plant system 702 may include an SMR system.

The first Sodium Formate (HCOONa) production system 704 may be configured to receive Sodium Hydroxide (NaOH) 714 (e.g., first Sodium Hydroxide (NaOH)) that may be produced via treatment of brine (e.g., treatment of brine produced by a desalination system) and Carbon Monoxide (CO) 716 produced by the pressure swing adsorption system 710 to produce Sodium Formate (HCOONa) 718. In various instances, the first Sodium Formate (HCOONa) production system 704 is by reacting Carbon Dioxide (CO) gas with dehydrated solid Sodium Hydroxide (NaOH) under pressure. The operating parameters is at 130° C. and a pressure range of 6-8 Bar. Production of Sodium Formate (HCOONa) 718 via the first Sodium Formate (HCOONa) production system 704 may be represented by the following reaction:

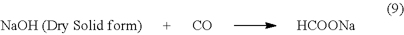

In various embodiments, the second Sodium Formate (HCOONa) production system 706 may be configured to receive Sodium Hydroxide (NaOH) (e.g., second Sodium Hydroxide (NaOH)), Sodium Bicarbonate (NaHCO₃), and Sodium Carbonate (Na₂CO₃) from the carbon capture process 720, and externally sourced Formic Acid (HCOOH) 722 to produce Sodium Formate (HCOONa) 724 and Carbon Dioxide (CO₂) 726. The Sodium Formate (HCOONa) 724 may be directed to the Hydrogen (H₂) production system 713. In various embodiments, the production of Sodium Formate (HCOONa) 724 via the second Sodium Formate (HCOONa) production system 706 using Formic Acid (HCOOH) from an external supply and a solution of Sodium Hydroxide (NaOH), Sodium Bicarbonate (NaHCO₃), and Sodium Carbonate (Na₂CO₃) from the carbon capture process 720 (e.g., DAC unit) may be represented by the following reactions:

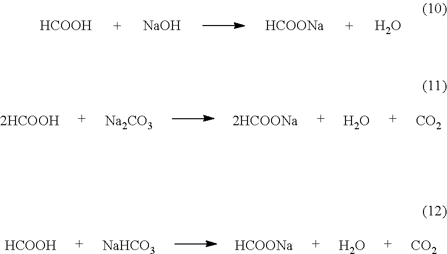

The Sodium Formate (HCOONa) 718 may be directed into the Hydrogen (H₂) production system 712. In some examples, the Hydrogen (H₂) production system 712 may have a first operating temperature range of 300° C. to 400° C. and a second operating temperature range of 450° C. to 800° C. to produce gas mixture 728. Production of gas mixture 728 via the Hydrogen (H₂) production system 712 may be represented by the following reactions:

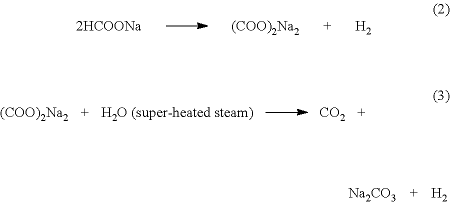

The gas mixture 728 may include Hydrogen (H₂) and Carbon Monoxide (CO), and the gas mixture 728 may be directed to the pressure swing adsorption system 710 for processing.

In some examples, the Hydrogen (H₂) production system 713 may be configured to receive process steam from the power plant system 702. For instance, the Hydrogen (H₂) production system 713 may include a first reaction chamber with a temperature in a range of 300° C. to 400° C. to facilitate the reaction represented by Equation (2). In various embodiments, the Hydrogen (H₂) production system 713 includes a second reaction chamber with a temperature in a range of 450° C. to 800° C. that is configured to receive the Sodium Oxalate ((COO)₂Na₂) from the first reaction chamber and process steam to facilitate a hydrothermal reaction. In various cases, the hydrothermal decomposition of Sodium Oxalate ((COO)₂Na₂) is represented by the following equation:

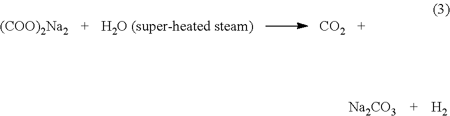

In some examples, the Hydrogen (H₂) production system 713 may have an operating temperature in a range of 450° C. to 800° C. to produce a mixture of Carbon Dioxide (CO₂) and Hydrogen (H₂). Production of the Carbon Dioxide (CO₂) and the Hydrogen (H₂) via the Hydrogen (H₂) production system 713 may be represented by the following equation:

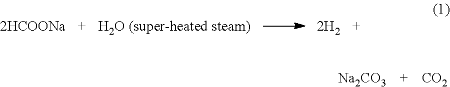

The Carbon Dioxide (CO₂) and the Hydrogen (H₂) produced by the Hydrogen (H₂) production system 713 may be directed to the pressure swing adsorption system 710 for processing.

In some embodiments, the SOEC stack 708 may include the Oxygen/anode side 730 and the fuel/cathode side 732. The Oxygen/anode side 730 may receive purge gas (e.g., Oxygen (O₂), atmospheric air, Nitrogen (N₂), etc.). The fuel/cathode side 732 may receive the Carbon Dioxide (CO₂) 726 produced by the second Sodium Formate (HCOONa) production system 706. The SOEC stack 708 may produce Oxygen (O₂) for use in hospitals, homes, and other industries. The SOEC stack 708 may produce gas mixture 734. In various examples, the gas mixture 734 may include Carbon Monoxide (CO) and/or Carbon Dioxide (CO₂). The gas mixture 734 may be directed to the pressure swing adsorption system 710.

The pressure swing adsorption system 710 may be configured to receive the gas mixture 734 from the SOEC stack 708, the gas mixture 728 from the Hydrogen (H₂) production system 712, and, in some cases, Carbon Dioxide (CO₂) and Hydrogen (H₂) from the Hydrogen (H₂) Production system 713. The pressure swing adsorption system 710 may produce the Carbon Monoxide (CO) 716, the Carbon Dioxide (CO₂) 736, and the Hydrogen (H₂) 738. The Carbon Monoxide (CO) 716 may be directed to the first Sodium Formate (HCOONa) production system 704 to be used to produce Sodium Formate (HCOONa) with dehydrated solid NaOH. The Carbon Dioxide (CO₂) 736 may be directed to the carbon capture process 720 to be recaptured and reused. The Hydrogen (H₂) 738 may be used to produce electricity (e.g., directed to a Hydrogen Fuel Cell) to help manage an EIM, and/or collected for storage (e.g., permanent tank, portable tank, etc.).

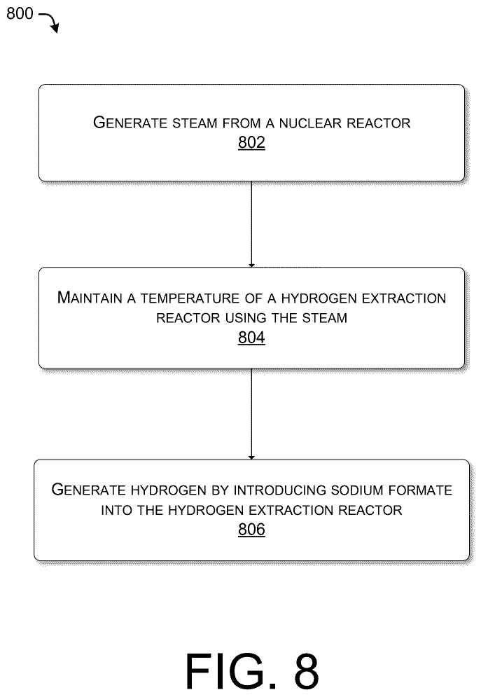

FIG. 8 illustrates a flow diagram of an example process 800 associated with utilizing a Hydrogen (H₂) extraction reactor (e.g., the Hydrogen (H₂) production system 114, the Hydrogen (H₂) extraction reactor 208, 304, or 434, or the like) for production of Hydrogen (H₂) from Sodium Formate (HCOONa). The example process 800 may include at least one of the power plant system 102, the power grid 104, the desalination system 106, the brine processing system 108, the direct air capture system 110, the sodium formate production system 112, the Hydrogen (H₂) production system 114, the hydrogen storage 116, and the Hydrogen Fuel Cell 118 described with reference to FIG. 1.

At 802, steam is generated by a nuclear reactor. In various examples, the nuclear reactor includes one or more small modular nuclear reactors. The nuclear reactor may include the power plant system 102 described with reference to FIG. 1. The nuclear reactor may be configured to generate the steam as a byproduct of the energy production process. According to some embodiments, the steam can be collected and transported from the nuclear reactor. For instance, the nuclear reactor may be located near (e.g., less than 1 km, less than 2 km, less than 5 km, etc.) a Hydrogen (H₂) extraction reactor that is configured to receive the steam from the nuclear reactor. Due to the proximity between the nuclear reactor and the Hydrogen (H₂) extraction reactor, the steam (e.g., the thermal energy of the steam) may be used to facilitate one or more thermal and/or hydrothermal processes by the Hydrogen (H₂) extraction reactor.

At 804, the steam is used to maintain an internal temperature of one or more reaction chambers of the Hydrogen (H₂) extraction reactor. For instance, the steam may be used to maintain the internal temperature of a reaction chamber within a particular range. In some examples, the steam may be introduced to the external surface of a reaction chamber of the Hydrogen (H₂) extraction reactor to facilitate a thermal process (e.g., a dry process). In some examples, the steam may be introduced into a reaction chamber of the Hydrogen (H₂) extraction reactor to facilitate a hydrothermal process (e.g., a wet process). According to various embodiments, the steam may be used to maintain the internal temperatures of one or more reaction chambers within distinct temperature ranges. In various cases, the steam generated by the nuclear reactor may be augmented by a thermal energy source. For instance, the steam may be heated using electrical power generated by the nuclear reactor. In some examples, the steam may be heated by a thermal recovery system that is powered by a power grid receiving electrical power from the nuclear reactor or a different energy source.

At 806, Sodium Formate (HCOONa) is introduced into the Hydrogen (H₂) extraction reactor. In some embodiments, the Sodium Formate (HCOONa) is hydrothermally decomposed using the steam generated by the nuclear reactor. The steam, in various examples, causes the reaction between the Sodium Formate (HCOONa) and water at a high temperature (e.g., above 450° C.) to form Hydrogen (H₂), Sodium Carbonate (Na₂CO₃), and Carbon Dioxide (CO₂). In some embodiments, the Sodium Formate (HCOONa) is thermally decomposed using steam generated by the nuclear reactor or using another thermal energy source. The steam, for instance, may be applied to an external surface of a reaction chamber of the Hydrogen (H₂) extraction reactor to increase an internal temperature of the reaction chamber without introducing water into the reaction chamber. The Sodium Formate (HCOONa) may decompose into Sodium Oxalate (COO)₂Na₂)) and Hydrogen (H₂) in a reaction chamber with an internal temperature in a range of 300° C. to 400° C. In some examples, the Hydrogen (H₂) extraction reactor includes an additional reaction chamber configured to receive the Sodium Oxalate (COO)₂Na₂)) and the steam generated by the nuclear reactor. The Sodium Oxalate (COO)₂Na₂)) may hydrothermally decompose in the presence of the steam to form Carbon Dioxide (CO₂), Sodium Carbonate (Na₂CO₃) 618, and Hydrogen (H₂). In various embodiments, the extracted gases (e.g., Carbon Dioxide (CO₂), Hydrogen (H₂), etc.) produced in the Hydrogen (H₂) extraction reactor may be applied to a system configured to separate the gases. For instance, the extracted gases may be applied to a pressure swing adsorption system configured to isolate Carbon Dioxide (CO₂) and Hydrogen (H₂).

In various embodiments, the Hydrogen (H₂) is provided to a fuel cell to produce electricity. The fuel cell is a Hydrogen Fuel Cell. In some examples, the Hydrogen (H₂) is stored before use by the fuel cell in electrical power production. For instance, the Hydrogen (H₂) can be provided to the fuel cell during peak time to support an EIM. In various embodiments, the fuel cell may use the Hydrogen (H₂) produce electrical power and supply the electrical power to a power grid.

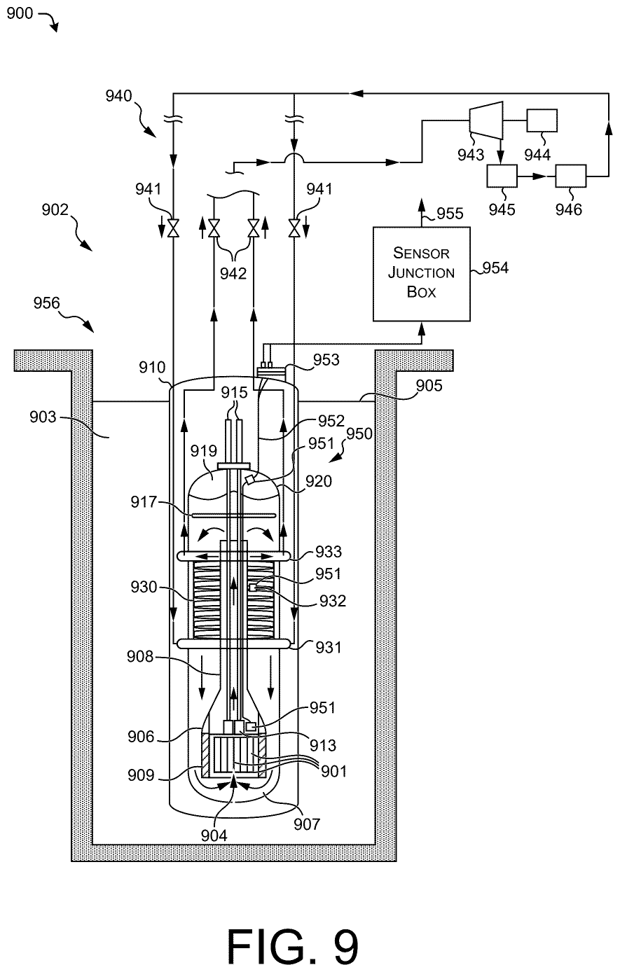

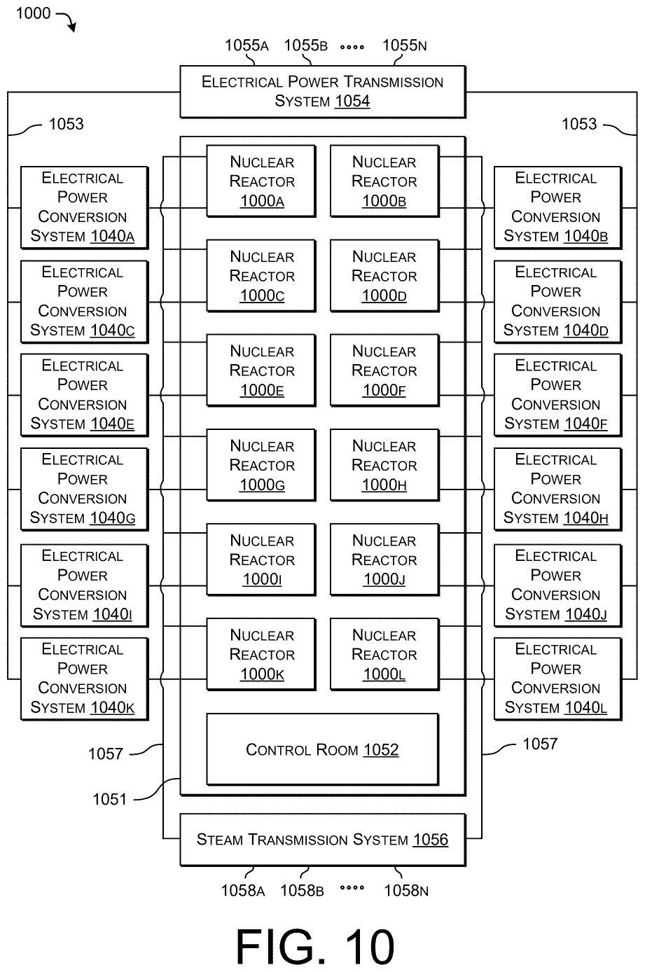

FIGS. 9 and 10 illustrate representative nuclear reactors that may be included in various embodiments of the present technology. FIG. 9 is a partially schematic, partially cross-sectional view of a nuclear reactor system 900 configured in accordance with embodiments of the present technology. The system 900 can include a power module 902 having a reactor core 904 in which a controlled nuclear reaction takes place. Accordingly, the reactor core 904 can include one or more fuel assemblies 901. The one or more fuel assemblies 901 can include fissile and/or other suitable materials. Heat from the reaction generates steam at a steam generator 930, which directs the steam to a power conversion system 940. The power conversion system 940 generates electrical power, and/or provides other useful outputs, such as super-heated steam. A sensor system 950 is used to monitor the operation of the power module 902 and/or other system components. The data obtained from the sensor system 950 can be used in real time to control the power module 902, and/or can be used to update the design of the power module 902 and/or other system components.

The power module 902 includes a containment vessel 910 (e.g., a radiation shield vessel, or a radiation shield container) that houses/encloses a reactor vessel 920 (e.g., a reactor pressure vessel, or a reactor pressure container), which in turn houses the reactor core 904. The containment vessel 910 can be housed in a power module bay 956. The power module bay 956 can contain a cooling pool 903 filled with water and/or another suitable cooling liquid. The bulk of the power module 902 can be positioned below a surface 905 of the cooling pool 903. Accordingly, the cooling pool 903 can operate as a thermal sink, for example, in the event of a system malfunction.

A volume between the reactor vessel 920 and the containment vessel 910 can be partially or completely evacuated to reduce heat transfer from the reactor vessel 920 to the surrounding environment (e.g., to the cooling pool 903). However, in other embodiments the volume between the reactor vessel 920 and the containment vessel 910 can be at least partially filled with a gas and/or a liquid that increases heat transfer between the reactor vessel 920 and the containment vessel 910. For example, the volume between the reactor vessel 920 and the containment vessel 910 can be at least partially filled (e.g., flooded with the primary coolant 907) during an emergency operation.

Within the reactor vessel 920, a primary coolant 907 conveys heat from the reactor core 904 to the steam generator 930. For example, as illustrated by arrows located within the reactor vessel 920, the primary coolant 907 is heated at the reactor core 904 toward the bottom of the reactor vessel 920. The heated primary coolant 907 (e.g., water with or without additives) rises from the reactor core 904 through a core shroud 906 and to a riser tube 908. The hot, buoyant primary coolant 907 continues to rise through the riser tube 908, then exits the riser tube 908 and passes downwardly through the steam generator 930. The steam generator 930 includes a multitude of conduits 932 that are arranged circumferentially around the riser tube 908, for example, in a helical pattern, as is shown schematically in FIG. 9. The descending primary coolant 907 transfers heat to a secondary coolant (e.g., water) within the conduits 932, and descends to the bottom of the reactor vessel 920 where the cycle begins again. The cycle can be driven by the changes in the buoyancy of the primary coolant 907, thus reducing or eliminating the need for pumps to move the primary coolant 907.

The steam generator 930 can include a feedwater header 931 at which the incoming secondary coolant enters the steam generator conduits 932. The secondary coolant rises through the conduits 932, converts to vapor (e.g., steam), and is collected at a steam header 933. The steam exits the steam header 933 and is directed to the power conversion system 940.

The power conversion system 940 can include one or more steam valves 942 that regulate the passage of high pressure, high temperature steam from the steam generator 930 to a steam turbine 943. The steam turbine 943 converts the thermal energy of the steam to electricity via a generator 944. The low-pressure steam exiting the turbine 943 is condensed at a condenser 945, and then directed (e.g., via a pump 946) to one or more feedwater valves 941. The feedwater valves 941 control the rate at which the feedwater re-enters the steam generator 930 via the feedwater header 931. In other embodiments, the steam from the steam generator 930 can be routed for direct use in an industrial process, such as a Hydrogen (H₂) and Oxygen (O₂) production plant, a chemical production plant, and/or the like, as described in detail below. Accordingly, steam exiting the steam generator 930 can bypass the power conversion system 940.

The power module 902 includes multiple control systems and associated sensors. For example, the power module 902 can include a hollow cylindrical reflector 909 that directs neutrons back into the reactor core 904 to further the nuclear reaction taking place therein. Control rods 913 are used to modulate the nuclear reaction and are driven via fuel rod drivers 915. The pressure within the reactor vessel 920 can be controlled via a pressurizer plate 917 (which can also serve to direct the primary coolant 907 downwardly through the steam generator 930) by controlling the pressure in a pressurizing volume 919 positioned above the pressurizer plate 917.

The sensor system 950 can include one or more sensors 951 positioned at a variety of locations within the power module 902 and/or elsewhere, for example, to identify operating parameter values and/or changes in parameter values. The data collected by the sensor system 950 can then be used to control the operation of the system 900, and/or to generate design changes for the system 900. For sensors positioned within the containment vessel 910, a sensor link 952 directs data from the sensors to a flange 953 (at which the sensor link 952 exits the containment vessel 910) and directs data to a sensor junction box 954. From there, the sensor data can be routed to one or more controllers and/or other data systems via a data bus 955.

FIG. 10 is a partially schematic, partially cross-sectional view of a nuclear reactor system 1000 configured in accordance with additional embodiments of the present technology. In some embodiments, the nuclear reactor system 1000 (“system 1000”) can include some features that are at least generally similar in structure and function, or identical in structure and function, to the corresponding features of the system 1000 described in detail above with reference to FIG. 10, and can operate in a generally similar or identical manner to the system 1000.

In the illustrated embodiment, the system 1000 includes a reactor vessel 1020 and a containment vessel 1010 surrounding/enclosing the reactor vessel 1020. In some embodiments, the reactor vessel 1020 and the containment vessel 1010 can be roughly cylinder-shaped or capsule-shaped. The system 1000 further includes a plurality of heat pipe layers 1011 within the reactor vessel 1020. In the illustrated embodiment, the heat pipe layers 1011 are spaced apart from and stacked over one another. In some embodiments, the heat pipe layers 1011 can be mounted/secured to a common frame 1012, a portion of the reactor vessel 1020 (e.g., a wall thereof), and/or other suitable structures within the reactor vessel 1020. In other embodiments, the heat pipe layers 1011 can be directly stacked on top of one another such that each of the heat pipe layers 1011 supports and/or is supported by one or more of the other ones of the heat pipe layers 1011.

In the illustrated embodiment, the system 1000 further includes a shield or reflector region 1014 at least partially surrounding a core region 1016. The heat pipe layers 1011 can be circular, rectilinear, polygonal, and/or can have other shapes, such that the core region 1016 has a corresponding three-dimensional shape (e.g., cylindrical, spherical). In some embodiments, the core region 1016 is separated from the reflector region 1014 by a core barrier 1015, such as a metal wall. The core region 1016 can include one or more fuel sources, such as fissile material, for heating the heat pipe layers 1011. The reflector region 1014 can include one or more materials configured to contain/reflect products generated by burning the fuel in the core region 1016 during operation of the system 1000. For example, the reflector region 1014 can include a liquid or solid material configured to reflect neutrons and/or other fission products radially inward toward the core region 1016. In some embodiments, the reflector region 1014 can entirely surround the core region 1016. In other embodiments, the reflector region 1014 may partially surround the core region 1016. In some embodiments, the core region 1016 can include a control material 1017, such as a moderator and/or coolant. The control material 1017 can at least partially surround the heat pipe layers 1011 in the core region 1016 and can transfer heat therebetween.

In the illustrated embodiment, the system 1000 further includes at least one heat exchanger 1030 (e.g., a steam generator) positioned around the heat pipe layers 1011. The heat pipe layers 1011 can extend from the core region 1016 and at least partially into the reflector region 1014 and are thermally coupled to the heat exchanger 1030. In some embodiments, the heat exchanger 1030 can be positioned outside of or partially within the reflector region 1014. The heat pipe layers 1011 provide a heat transfer path from the core region 1016 to the heat exchanger 1030. For example, the heat pipe layers 1011 can each include an array of heat pipes that provide a heat transfer path from the core region 1016 to the heat exchanger 1030. When the system 1000 operates, the fuel in the core region 1016 can heat and vaporize a fluid within the heat pipes in the heat pipe layers 1011, and the fluid can carry the heat to the heat exchanger 1030. The heat pipes in the heat pipe layers 1011 can then return the fluid toward the core region 1016 via wicking, gravity, and/or other means to be heated and vaporized once again.

In some embodiments, the heat exchanger 1030 can be similar to the steam generator 930 of FIG. 9 and, for example, can include one or more helically-coiled tubes that wrap around the heat pipe layers 1011. The tubes of the heat exchanger 1030 can include or carry a working fluid (e.g., a coolant such as water or another fluid) that carries the heat from the heat pipe layers 1011 out of the reactor vessel 1020 and the containment vessel 1010 for use in generating electricity, steam, and/or the like. For example, in the illustrated embodiment the heat exchanger 1030 is operably coupled to a turbine 1043, a generator 1044, a condenser 1045, and a pump 1046. As the working fluid within the heat exchanger 1030 increases in temperature, the working fluid may begin to boil and vaporize. The vaporized working fluid (e.g., steam) may be used to drive the turbine 1043 to convert the thermal potential energy of the working fluid into electrical energy via the generator 1044. The condenser 1045 can condense the working fluid after it passes through the turbine 1043, and the pump 1046 can direct the working fluid back to the heat exchanger 1030 where it can begin another thermal cycle. In other embodiments, steam from the heat exchanger 1030 can be routed for direct use in an industrial process, such as an enhanced oil recovery operation described in detail below. Accordingly, steam exiting the heat exchanger 1030 can bypass the turbine 1043, the generator 1044, the condenser 1045, the pump 1046, etc.

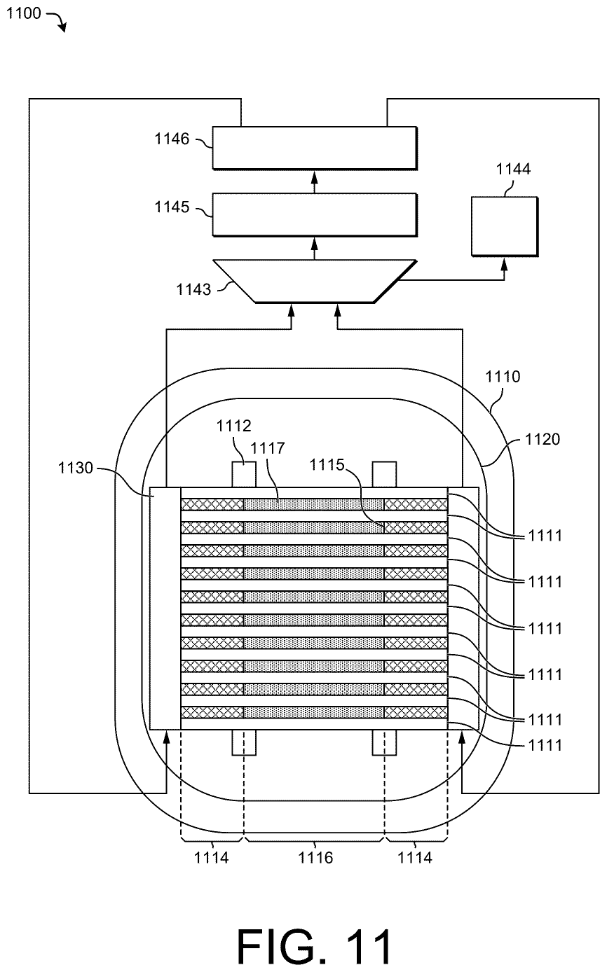

FIG. 11 is a schematic view of a nuclear power plant system 1150 including multiple nuclear reactors 1100 in accordance with embodiments of the present technology. Each of the nuclear reactors 1100 (individually identified as first through twelfth nuclear reactors 1100a-l, respectively) can be similar to or identical to the nuclear reactor 1100 and/or the nuclear reactor 1100 described in detail above with reference to FIGS. 9 and 10. The power plant system 1150 (“power plant system 1150”) can be “modular” in that each of the nuclear reactors 1100 can be operated separately to provide an output, such as electricity or steam. The power plant system 1150 can include fewer than twelve of the nuclear reactors 1100 (e.g., two, three, four, five, six, seven, eight, nine, ten, or eleven of the nuclear reactors 1100), or more than twelve of the nuclear reactors 1100. The power plant system 1150 can be a permanent installation or can be mobile (e.g., mounted on a truck, tractor, mobile platform, and/or the like). In the illustrated embodiment, each of the nuclear reactors 1100 can be positioned within a common housing 1151, such as a reactor plant building, and controlled and/or monitored via a control room 1152.

Each of the nuclear reactors 1100 can be coupled to a corresponding electrical power conversion system 1140 (individually identified as first through twelfth electrical power conversion systems 1140a-1, respectively). The electrical power conversion systems 1140 can include one or more devices that generate electrical power or some other form of usable power from steam generated by the nuclear reactors 1100. In some embodiments, multiple ones of the nuclear reactors 1100 can be coupled to the same one of the electrical power conversion systems 1140 and/or one or more of the nuclear reactors 1100 can be coupled to multiple ones of the electrical power conversion systems 1140 such that there is not a one-to-one correspondence between the nuclear reactors 1100 and the electrical power conversion systems 1140.

The electrical power conversion systems 1140 can be further coupled to an electrical power transmission system 1154 via, for example, an electrical power bus 1153. The electrical power transmission system 1154 and/or the electrical power bus 1153 can include one or more transmission lines, transformers, and/or the like for regulating the current, voltage, and/or other characteristic(s) of the electricity generated by the electrical power conversion systems 1140. The electrical power transmission system 454 can route electricity via a plurality of electrical output paths 1155 (individually identified as electrical output paths 1155a-n) to one or more end users and/or end uses, such as different electrical loads of an integrated energy system.

Each of the nuclear reactors 1100 can further be coupled to a steam transmission system 1156 via, for example, a steam bus 1157. The steam bus 1157 can route steam generated from the nuclear reactors 1100 to the steam transmission system 1156 which in turn can route the steam via a plurality of steam output paths 1158 (individually identified as steam output paths 1158a-n) to one or more end users and/or end uses, such as different steam inputs of an integrated energy system.

In some embodiments, the nuclear reactors 1100 can be individually controlled (e.g., via the control room 1152) to provide steam to the steam transmission system 1156 and/or steam to the corresponding one of the electrical power conversion systems 1140 to provide electricity to the electrical power transmission system 1154. In some embodiments, the nuclear reactors 1100 are configured to provide steam either to the steam bus 1157 or to the corresponding one of the electrical power conversion systems 1140 and can be rapidly and efficiently switched between providing steam to either. Accordingly, in some aspects of the present technology the nuclear reactors 1100 can be modularly and flexibly controlled such that the power plant system 1150 can provide differing levels/amounts of electricity via the electrical power transmission system 1154 and/or steam via the steam transmission system 1156. For example, where the power plant system 1150 is used to provide electricity and steam to one or more industrial process-such as various components of the integrated energy systems, the nuclear reactors 1100 can be controlled to meet the differing electricity and steam requirements of the industrial processes.

As one example, during a first operational state of an integrated energy system employing the power plant system 1150, a first subset of the nuclear reactors 1100 (e.g., the first through sixth nuclear reactors 1100a-f) can be configured to provide steam to the steam transmission system 1156 for use in the first operational state of the integrated energy system, while a second subset of the nuclear reactors 1100 (e.g., the seventh through twelfth nuclear reactors 1100g-1) can be configured to provide steam to the corresponding ones of the electrical power conversion systems 1140 (e.g., the seventh through twelfth electrical power conversion systems 1140g-1) to generate electricity for the first operational state of the integrated energy system. Then, during a second operational state of the integrated energy system when a different (e.g., greater or lesser) amount of steam and/or electricity is required, some or all the first subset of the nuclear reactors 1100 can be switched to provide steam to the corresponding ones of the electrical power conversion systems 1140 (e.g., the seventh through twelfth electrical power conversion systems 1140g-1) and/or some or all of the second subset of the nuclear reactors 1100 can be switched to provide steam to the steam transmission system 1156 to vary the amount of steam and electricity produced to match the requirements/demands of the second operational state. Other variations of steam and electricity generation are possible based on the needs of the integrated energy system. That is, the nuclear reactors 1100 can be dynamically/flexibly controlled during other operational states of an integrated energy system to meet the steam and electricity requirements of the operational state.

In contrast, some conventional nuclear power plant systems can typically generate either steam or electricity for output and cannot be modularly controlled to provide varying levels of steam and electricity for output. Moreover, it is typically difficult (e.g., expensive, time consuming, etc.) to switch between steam generation and electricity generation in conventional nuclear power plant systems. Specifically, for example, it is typically extremely time consuming to switch between steam generation and electricity generation in prototypical large nuclear power plant systems.

The nuclear reactors 1100 can be individually controlled via one or more operators and/or via a computer system. Accordingly, many embodiments of the technology described herein may take the form of computer- or machine- or controller-executable instructions, including routines executed by a programmable computer or controller. Those skilled in the relevant art will appreciate that the technology can be practiced on computer/controller systems other than those shown and described herein. The technology can be embodied in a special-purpose computer, controller or data processor that is specifically programmed, configured, or constructed to perform one or more of the computer-executable instructions described below. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like). Information handled by these computers can be presented at any suitable display medium, including a liquid crystal display (LCD).

The technology can also be practiced in distributed environments, where tasks or modules are performed by remote processing devices that are linked through a communications network. In a distributed computing environment, program modules or subroutines may be located in local and remote memory storage devices. Aspects of the technology described herein may be stored or distributed on computer-readable media, including magnetic or optically readable or removable computer disks, as well as distributed electronically over networks. Data structures and transmissions of data particular to aspects of the technology are also encompassed within the scope of the embodiments of the technology.

### CONCLUSION

Each of the references cited herein is incorporated herein by reference in its entirety.

The above detailed description of embodiments of the present technology are not intended to be exhaustive or to limit the technology to the precise forms disclosed above. Although specific embodiments of, and examples for, the technology are described above for illustrative purposes, various equivalent modifications are possible within the scope of the technology, as those skilled in the relevant art will recognize. For example, although steps may be presented in a given order, in other embodiments, the steps may be performed in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. An integrated energy system comprising:
a power plant including at least one nuclear reactor, the at least one nuclear reactor being configured to generate steam;
a Sodium Formate (HCOONa) production process configured to produce Sodium Formate (HCOONa);
a Hydrogen (H₂) extraction reactor configured to:
receive the Sodium Formate (HCOONa) and the steam generated by the at least one nuclear reactor; and
generate extracted gases including Hydrogen (H₂), wherein the steam is used to maintain a temperature of at least one reaction chamber of the Hydrogen (H₂) extraction reactor within a range; and
an input device for introducing the Sodium Formate (HCOONa) into the Hydrogen (H₂) extraction reactor at a steady rate.

2. The integrated energy system of claim 1, wherein the at least one reaction chamber includes a first reaction chamber configured to receive the Sodium Formate (HCOONa) and produce Sodium Oxalate ((COO)₂Na₂) and Hydrogen (H₂), and wherein the steam is used to maintain a temperature of the first reaction chamber within a range of 300° C. to 400° C.

3. The integrated energy system of claim 2, wherein the at least one reaction chamber further includes a second reaction chamber configured to:
receive the Sodium Oxalate ((COO)₂Na₂) and the steam generated by the at least one nuclear reactor; and
produce Sodium Carbonate (Na₂CO₃), Carbon Dioxide (CO₂), and Hydrogen (H₂), and wherein the steam is used to maintain a temperature of the second reaction chamber within a range of 450° C. to 800° C.

4. The integrated energy system of claim 3, wherein the input device includes a first rotating spiral configured to direct the Sodium Formate (HCOONa) into an upper portion of the first reaction chamber,
wherein the first reaction chamber includes a second rotating spiral configured to direct Sodium Oxalate ((COO)₂Na₂) out of a lower portion of the first reaction chamber, and
wherein the second reaction chamber includes:
a third rotating spiral configured to direct the Sodium Oxalate ((COO)₂Na₂) into an upper portion of the second reaction chamber; and
a fourth rotating spiral configured to direct the Sodium Carbonate (Na₂CO₃) out of a lower portion of the second reaction chamber.

5. The integrated energy system of claim 1, wherein the at least one reaction chamber is configured to:
receive the Sodium Formate (HCOONa) and the steam generated by the at least one nuclear reactor; and
produce Hydrogen (H₂), Carbon Dioxide (CO₂), and Sodium Carbonate (Na₂CO₃), and wherein the steam is used to maintain the temperature of the at least one reaction chamber within a range of 450° C. and 800° C.

6. The integrated energy system of claim 1, further comprising:
a desalination system configured to receive at least a portion of electricity generated by the power plant and at least a portion of the steam to produce brine and water;
an electrolysis process configured to process the brine into Sodium Hydroxide (NaOH); and
a carbon capture process configured to receive the Sodium Hydroxide (NaOH) to produce a solution containing at least Sodium Hydroxide (NaOH), Sodium Bicarbonate (NaHCO₃), and Sodium Carbonate (Na₂CO₃),
wherein the Sodium Formate (HCOONa) production process is configured to combine the solution containing at least Sodium Hydroxide (NaOH), Sodium Bicarbonate (NaHCO₃), and Sodium Carbonate (Na₂CO₃) with Formic Acid (HCOOH) to produce Sodium Formate (HCOONa) and Carbon Dioxide (CO₂).

7. The integrated energy system of claim 1, further comprising: a fuel cell configured to receive the Hydrogen (H₂).

8. The integrated energy system of claim 7, wherein the fuel cell is a Hydrogen Fuel Cell, the Hydrogen Fuel Cell including a thermal recovery system configured to transfer heat from the Hydrogen Fuel Cell to the Hydrogen (H₂) extraction reactor.

9. The integrated energy system of claim 1, wherein the Hydrogen (H₂) extraction reactor is further configured to produce extracted gases and further comprising a pressure swing adsorption system configured to separate the extracted gases into at least one of Hydrogen (H₂) and Carbon Dioxide (CO₂).

10. The integrated energy system of claim 1, wherein the Hydrogen (H₂) is collected in a storage tank.

11. A method, comprising:
producing steam using at least one nuclear reactor;
maintaining, using the steam produced by the at least one nuclear reactor, a temperature of at least one reaction chamber of a Hydrogen (H₂) extraction reactor within a range; and
producing Hydrogen (H₂) utilizing the Hydrogen (H₂) extraction reactor configured to receive Sodium Formate (HCOONa) from a Sodium Formate (HCOONa) production process.

12. The method of claim 11, wherein the at least one nuclear reactor includes a small modular nuclear reactor power plant system.

13. The method of claim 11, wherein producing Hydrogen (H₂) utilizing the Hydrogen (H₂) extraction reactor comprises:
producing Sodium Oxalate ((COO)₂Na₂) and Hydrogen (H₂) by introducing the Sodium Formate (HCOONa) into a first reaction chamber of the Hydrogen (H₂) extraction reactor, a temperature of the first reaction chamber being maintained in a range of 300° C. to 400° C. using the steam.

14. The method of claim 13, wherein producing Hydrogen (H₂) utilizing the Hydrogen (H₂) extraction reactor further comprises:
producing Sodium Carbonate (Na₂CO₃), Carbon Dioxide (CO₂), and Hydrogen (H₂) by introducing the Sodium Oxalate ((COO)₂Na₂) and the steam from the at least one nuclear reactor into a second reaction chamber of the Hydrogen (H₂) extraction reactor, a temperature of the second reaction chamber being maintained in a range of 450° C. to 800° C. using the steam.

15. The method of claim 11, wherein producing Hydrogen (H₂) utilizing the Hydrogen (H₂) extraction reactor comprises:
producing Hydrogen (H₂), Sodium Carbonate (Na₂CO₃), and Carbon Dioxide (CO₂) by introducing the Sodium Formate (HCOONa) and the steam from the at least one nuclear reactor into the at least one reaction chamber of the Hydrogen (H₂) extraction reactor, wherein the temperature of the at least one reaction chamber is maintained in a range of 450° C. to 800° C. using the steam.

16. The method of claim 11, wherein the Hydrogen (H₂) extraction reactor is further configured to produce extracted gases, the method further comprising:
separating the extracted gases into at least one of Hydrogen (H₂) and Carbon Dioxide (CO₂).

17. The method of claim 11, further comprising: storing the Hydrogen (H₂) produced by the Hydrogen (H₂) extraction reactor.

18. The method of claim 11, further comprising: producing electricity utilizing a fuel cell configured to receive the Hydrogen (H₂) to produce electricity.

19. The method of claim 18, further comprising: heating the steam produced by the at least one nuclear reactor by:
directing thermal energy generated by a thermal recovery system of the fuel cell to the Hydrogen (H₂) extraction reactor.

20. The method of claim 11, further comprising:
producing brine utilizing a desalination system configured to receive the steam;
producing Sodium Hydroxide (NaOH) by processing the brine via an electrolysis process;
producing a solution including at least Sodium Hydroxide (NaOH), Sodium Bicarbonate (NaHCO₃), and Sodium Carbonate (Na₂CO₃) utilizing a carbon capture process configured to receive the Sodium Hydroxide (NaOH); and
producing Carbon Dioxide (CO₂) and Sodium Formate (HCOONa) utilizing the solution and Formic Acid (HCOOH).
